(19) United States
(12) Patent Application Publication
(10) Pub. No.: US 2025/0090618 A1
(43) Pub. Date: Mar. 20, 2025

(54) COMBINATION FOR USE FOR THE TREATMENT OF HYPERCHOLESTEROLEMIA, HYPERLIPIDEMIA, CARDIOVASCULAR DISEASE AND METABOLIC SYNDROME

(71) Applicant: MEDA PHARMA S.p.A., MONZA (IT)

(72) Inventors: ANDREA ZANARDI, MONZA (IT); ELENA GELFI; MANUEL MOSCONI, MONZA (IT); FRANCO GASPARRI, MONZA (IT)

(21) Appl. No.: 18/580,373

(22) PCT Filed: Jul. 19, 2022
(86) PCT No.: PCT/EP2022/070248

§ 371 Date: Jan. 18, 2024

(30) Foreign Application Priority Data
Jul. 19, 2021 (GB) 2110358.5

(51) Int. Cl. A61K 36/752; A61K 31/575; A61K 31/7048; A61K 36/15; A61K 45/06

(52) U.S. Cl. CPC A61K 36/752; A61K 31/575; A61K 31/7048; A61K 36/15; A61K 45/06

(57) ABSTRACT

The invention relates to a combination of: —naringin and —stanols for use in the treatment or prevention of hypercholesterolemia. The invention also relates to compositions comprising the combination.

This invention relates to new compositions, in particular nutraceutical compositions, and their uses, in particular for the treatment or prevention of hypercholesterolemia, hyperlipidemia, cardiovascular disease and metabolic syndrome.

### BACKGROUND OF THE INVENTION

Hypercholesterolemia is a well-known risk factor for coronary artery, cerebrovascular and peripheral artery diseases. In fact, any reduction of basal cholesterol in plasma levels is correlated to a proportionally reduced incidence of cardiovascular complications (myocardial infarction, stroke, peripheral obstructive arterial disease). The correlation already exists before the first clinical event, relevant for primary prevention, as well as for the cardiovascular events that follow the first clinical vent, relevant for secondary prevention.

Although hypercholesterolemia itself is asymptomatic, longstanding elevation of serum cholesterol can lead to atherosclerosis. Atherosclerosis, the hardening of arteries under oxidative stress is related to oxidative changes of low density lipoproteins (LDL). The antioxidants prevent the formation of oxidized LDL during atherogenesis. Perhaps more than one mechanism is involved in the atherosclerosis disease where LDL is oxidized in all the cells of arterial wall during the development of this disease. The oxidation of LDL produces lipid peroxidation products such as isoprostans from arachidonic, eicosapentaenoic and docosahexaenoic acids, oxysterols from cholesterol, hydroxyl fatty acids, lipid peroxides and aldehydes. The lipid peroxidation bioassay can serve as a marker for the risk of cardiovascular disease.

The treatment of hypercholesterolemia with specific drugs is recommended in secondary prevention. Conversely, there is no rationale for the lifetime use of hypocholesterolemic drugs in primary prevention, i.e. in subjects without or with marginal cardiovascular symptoms, in whom therapeutic life-style changes (TLC) appear to be better practicable and effective. TLC are important actions to control hypercholesterolemia. In particular, diet management can combine supplements with improved compliance with an appropriate dietary regimen. However, to be effective in primary prevention, TLC must be followed without derogations for the whole life, an extremely difficult task in the present social conditions.

Accordingly, it is an object of the present invention to provide further methods that can manage and treat these chronic diseases with natural products and food supplements which are able to achieve a satisfactory control of cardiovascular risk factors in primary prevention.

### SUMMARY OF THE INVENTION

In a first aspect, the invention provides a combination of:
naringin and
stanols

for use in the treatment or prevention of hypercholesterolemia, hyperlipidemia, cardiovascular disease and metabolic syndrome.

The invention also provides a method for the treatment or prevention of hypercholesterolemia, hyperlipidemia, cardiovascular disease and metabolic syndrome, comprising administering to a human a combination of:
naringin and
stanols.

The invention also provides the use of a combination of:
naringin and
stanols

for the manufacture of a medicament or nutraceutical for the treatment or prevention of hypercholesterolemia, hyperlipidemia, cardiovascular disease and metabolic syndrome,

In a second aspect, the invention provides a combination of:
Citrus bergamia extract (standardized in naringin 15%) and
Pine tree extract (standardized in stanols 10%)

for use in the treatment or prevention of hypercholesterolemia, hyperlipidemia, cardiovascular disease and metabolic syndrome.

The invention also provides a method for the treatment or prevention of hypercholesterolemia, hyperlipidemia, cardiovascular disease and metabolic syndrome comprising administering to a human a combination of:
Citrus bergamia extract (standardized in naringin 15%) and
Pine tree extract (standardized in stanols 10%).

The invention also provides the use of a combination of:
Citrus bergamia extract (standardized in naringin 15%) and
Pine tree extract (standardized in stanols 10%)

for the manufacture of a medicament or nutraceutical for the treatment or prevention of hypercholesterolemia, hyperlipidemia, cardiovascular disease or metabolic syndrome.

The combination of the invention is for use in the treatment or prevention of hypercholesterolemia, hyperlipidemia, cardiovascular disease or metabolic syndrome. Thus, the combination may be administered as a prophylactic treatment to prevent the condition developing, or to treat the condition after it has already developed.

Surprisingly the applicant has found that the combination of Citrus bergamia extract comprising naringin and the Pine tree extract comprising stanols is useful for the treatment or prevention of hypercholesterolemia, hyperlipidemia, cardiovascular disease and metabolic syndrome.

In all aspects of the invention, the stanols preferably comprise at least beta sitostanol and/or campestanol.

### Definitions

The proportions of the various components of the combination are defined relative to other components. The wt % (weight percent) of a particular component, based on the other components, is the weight (mass) of the particular component, divided by the weight (mass) of based on weight of the botanical extract, times 100 i.e.

$$wt \% single component X ( based on weight of the botanical extract Y ) = \frac{w t ( X )}{w t ( Y )} \times 100$$

Bergamot, the common name of Citrus bergamia Risso & Poiteau, belongs to the family Rutaceae, subfamily Esperidea and it has been widespread in the Mediterranean area for centuries. The tree Citrus bergamia is found in the Calabria region specifically, due to its unique climate that is suitable for its growth.

Pine tree extract is prepared from Tall Oil Pitch, a side steam recovered from trees, mainly Pinus elliotii and Pinus taeda, used in the pull and paper industry containing stanols, especially sitostanol and campestanol,

### DETAILED DESCRIPTION OF THE INVENTION

In a first aspect, the invention provides a combination of:
naringin and
stanols

for use in the treatment or prevention of hypercholesterolemia, hyperlipidemia, cardiovascular disease or metabolic syndrome.

The invention also provides naringin for use in the treatment or prevention of hypercholesterolemia, hyperlipidemia, cardiovascular disease or metabolic syndrome, wherein the naringin is administered in combination with stanols.

The invention also provides stanols for use in the treatment or prevention of hypercholesterolemia, hyperlipidemia, cardiovascular disease or metabolic syndrome, wherein the stanols are administered in combination with naringin.

In a second aspect, the invention provides a combination of:
Citrus bergamia extract (standardized in naringin 15%) and
Pine tree extract (standardized in stanols 10%)

for use in the treatment or prevention of hypercholesterolemia, hyperlipidemia, cardiovascular disease or metabolic syndrome.

### Naringin

Naringin is bitter-tasting flavanone-7-O-glycoside between the flavanone naringenin and the disaccharide neohesperidose. It has the chemical name 7-[[2-O-(6-Deoxy-α-L-mannopyranosyl)-ß-Dglucopyranosyl]oxy]-2,3-dihydro-5-hydroxy-2-(4-hydroxyphenyl)-4H-1-benzopyran-4-one, and has the following structure:

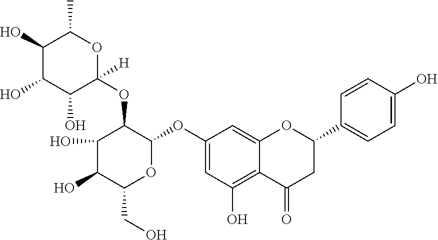 [SMILES: C[C@@H]1O[C@@H](O[C@H]2[C@H](Oc3cc(O)c4c(c3)O[C@H](c3ccc(O)cc3)CC4=O)O[C@H](CO)[C@@H](O)[C@@H]2O)[C@H](O)[C@H](O)[C@H]1O]

Naringin can be extracted from Citrus bergamia, Citrus paradisi, Citrus sulcata, Citrus aurantium, Citrus sinensis or Citrus erythrosa (see M. Yano et. al., J. Agric Food Chem 1999, 47, 128-135; Tables 1 and 2).

### Neoeriocitrin and Neohesperidin

Neoeriocitrin is a 7-O-glycoside of the flavanone eriodictyol and the disaccharide neohesperidose. It has the chemical name (S)-3′,4′,5,7-Tetrahydroxyflavanone-7-[2-O-(α-L-rhamnopyranosyl)-ß-D-glucopyranoside], and has the following structure:

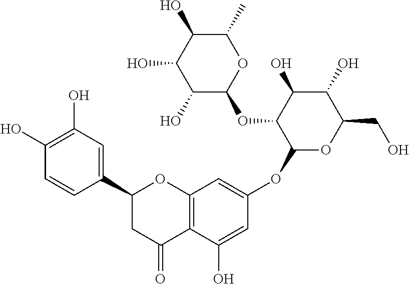

Neohesperidin is the 7-O-neohesperidose derivative of hesperetin, and has the following structure:

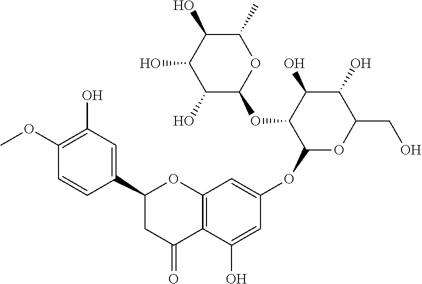

Neoeriocitrin and neohesperidin can also be extracted from Citrus bergamia, Citrus paradisi, Citrus sulcata, Citrus aurantium, Citrus sinensis or Citrus erythrosa (see M. Yano et. al., J. Agric Food Chem 1999, 47, 128-135; Tables 1 and 2). Neoeriocitrin and neohesperidin may be present in various embodiments, in particular in combinations used in the present invention.

### Stanols

Stanols are botanical compounds often found in small amounts in plant oils.

Beta sitostanol has the chemical name (3S,5S,8R,9S,10S,13R,14S,17R)-17-((2R,5R)-5-ethyl-6-methylheptan-2-yl)-10,13-dimethylhexadecahydro-1H-cyclopenta[a]phenanthren-3-ol, and has the following structure:

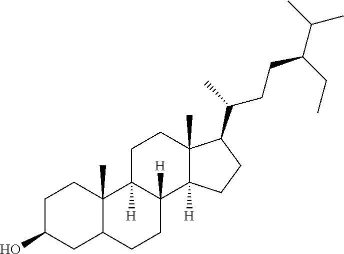

Campestanol has the chemical name (3β,5α,24R)-Ergostan-3-ol and has the following structure:

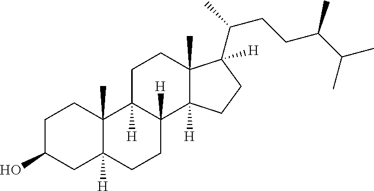

Stanols are extracted exclusively from Tall Oil Pitch, a side steam recovered from trees, mainly Pinus elliotii and Pinus taeda, used in the pull and paper industry.

(see R. Moreau, Journal of A International Vol. 98, No. 3, 2015, FIG. 1 and table 2)

### The Combination

In one embodiment, the combination used in the present invention includes:
naringin and
stanols.

In one embodiment, the wt ratio of naringin to stanols is selected from:
10:1 to 1:10
9:1 to 1:9;
8:1 to 1:8;
7:1 to 1:7;
6:1 to 1:6;
5:1 to 1:5;
4:1 to 1:4;
3:1 to 1:3; and
2:1 to 1:2.

preferably wherein the wt ratio of naringin to stanols is 2:1 to 1:2.

In one embodiment, the wt ratio of naringin to stanols is selected from:
1:1 to 1:10;
1:1 to 1:9;
1:1 to 1:8;
1:1 to 1:7;
1:1 to 1:6;
1:1 to 1:5;
1:1 to 1:4;
1:1 to 1:3; and
1:1 to 1:2.

In one embodiment, the wt ratio of naringin to stanols is selected from:
10:1 to 1:1;
9:1 to 1:1;
8:1 to 1:1;
7:1 to 1:1;
6:1 to 1:1;
5:1 to 1:1;
4:1 to 1:1;
3:1 to 1:1; and
2:1 to 1:1.

In one embodiment, the combination used in the present invention includes:
Citrus bergamia extract; (standardized in naringin 15%) and
Pine tree extract (standardized in stanols 10%),

preferably the Citrus bergamia extract and Pine tree extract being prepared as described in example 1.

In one embodiment, the weight ratio of the Citrus bergamia extract (standardized in naringin 15%) to the Pine tree extract (standardized in stanols 10%) is selected from:
10:1 to 1:10;
9:1 to 1:9;
8:1 to 1:8;
7:1 to 1:7;
6:1 to 1:6;
5:1 to 1:5;
4:1 to 1:4;
3:1 to 1:3; and
2:1 to 1:2.

preferably wherein the weight ratio of the Citrus bergamia extract (standardized in naringin 15%) to the Pine tree extract (standardized in stanols 10%) is 2:1 to 1:2.

In one embodiment, the weight ratio of the Citrus bergamia extract (standardized in naringin 15%) to the Pine tree extract (standardized in stanols 10%) is selected from:
1:1 to 1:10;
1:1 to 1:9;
1:1 to 1:8;
1:1 to 1:7;
1:1 to 1:6;
1:1 to 1:5;
1:1 to 1:4;
1:1 to 1:3; and
1:1 to 1:2.

In one embodiment, the weight ratio of the Citrus bergamia extract (standardized in naringin 15%) to the Pine tree extract (standardized in stanols 10%) is selected from:
10:1 to 1:1;
9:1 to 1:1;
8:1 to 1:1;
7:1 to 1:1;
6:1 to 1:1;
5:1 to 1:1;
4:1 to 1:1;
3:1 to 1:1; and
2:1 to 1:1.

In one embodiment of the invention, the combination includes administration of HMG-CoA reductase inhibitors such as Fermented Red Rice (also known as red yeast rice), comprising monacolin K, and/or other botanical extracts for metabolic syndrome and cholesterolemia management such as one or more botanical extract selected from the list comprising Berberis aristata (Berberine), Cynara scolymus extract, Cynara cardunculus extract, Allium sativum, Salvia miltiorrhiza, Policosanol, Green tea (Camellia sinensis) extract, Annur apple (Melannurca campana) extract, Curcuma longa and curcuminoids, Spirulina, Chitosan, Betaglucan, and Glucomannan.

In one embodiment of the invention, the combination includes administration of one or more other active substances such as Coenzyme Q10, Astaxantine, Folic acid, and/or Orthosiphon extract.

In one embodiment of the invention, the composition does not include tocotrienols, hydroxytyrosol, or an extract comprising hydroxytyrosol.

### The Use in Combination

The combinations of the invention may produce an increased therapeutic effect relative to the therapeutic effect of the individual components when administered alone.

In particular, the combination may, relative to the individual components when administered alone, provide an additive effect or a synergistic effect.

A “synergistic” effect occurs when the combination provides an effect which is larger than the sum of the therapeutic effects of the agents administered alone.

An “additive” effect occurs when the combination provides an effect which is larger than the either of the components when administered alone.

The term “combination” means that the components are administered as part of the same overall treatment regimen.

The components may be administered at the same time or at different times. It will therefore be appreciated that the components of the combination may be administered sequentially (e.g. before or after) or simultaneously, either in the same formulation (i.e. together), or in different formulations (i.e. separately).

In one embodiment, the components are administered simultaneously in the same formulation i.e. a unitary formulation comprising all components in the same dose.

In one embodiment, the components are administered simultaneously in different formulations. In one embodiment, the components are administered separately or sequentially in different formulations.

### Compositions

In another aspect, the invention provides a pharmaceutical or nutraceutical composition comprising a combination of:
naringin and
stanols

and a pharmaceutically or nutraceutically acceptable excipient, wherein the composition does not include tocotrienols, hydroxytyrosol, or an extract comprising hydroxytyrosol.

In another aspect, the invention provides a pharmaceutical or nutraceutical composition comprising a combination of:
naringin and
stanols

and a pharmaceutically or nutraceutically acceptable excipient, wherein the naringin and the stanols are present at from 0.1 to 750 mg, 1 to 225 mg, 2 to 120 mg, 5 to 75 mg, 2 to 30 mg, or 10 to 150 mg.

The invention provides a pharmaceutical or nutraceutical composition comprising a combination of:
naringin and
stanols

and a pharmaceutically or nutraceutically acceptable excipient, wherein the weight ratio of naringin to stanols is selected from:
10:1 to 1:10;
9:1 to 1:9;
8:1 to 1:8;
7:1 to 1:7;
6:1 to 1:6;
5:1 to 1:5;
4:1 to 1:4;
3:1 to 1:3; and
2:1 to 1:2.

preferably wherein the wt ratio of naringin to stanols is 2:1 to 1:2.

The invention provides a pharmaceutical or nutraceutical composition comprising a combination of:
naringin and
stanols

and a pharmaceutically or nutraceutically acceptable excipient, wherein the weight ratio of naringin to stanols is selected from:
1:1 to 1:10;
1:1 to 1:9;
1:1 to 1:8;
1:1 to 1:7;
1:1 to 1:6;
1:1 to 1:5;
1:1 to 1:4;
1:1 to 1:3; and
1:1 to 1:2.

The invention provides a pharmaceutical or nutraceutical composition comprising a combination of:
Citrus bergamia extract (standardized in naringin 15%) and
Pine tree extract (standardized in stanols 10%)

and a pharmaceutically or nutraceutically acceptable excipient, wherein the composition does not include tocotrienols, hydroxytyrosol, or an extract comprising hydroxytyrosol

The invention provides a pharmaceutical or nutraceutical composition comprising a combination of:
Citrus bergamia extract (standardized in naringin 15%) and
Pine tree extract (standardized in stanols 10%)

and a pharmaceutically or nutraceutically acceptable excipient, wherein Citrus bergamia extract (standardized in naringin 15%) to Pine tree extract (standardized in stanols 10%) are present at from 0.1 to 5000 mg, 1 to 1500 mg, 2 to 800 mg, 5 to 500 mg, 2 to 200 mg, or 10 to 1000 mg.

The invention provides a pharmaceutical or nutraceutical composition comprising a combination of:
Citrus bergamia extract (standardized in naringin 15%) and
Pine tree extract (standardized in stanols 10%)

and a pharmaceutically or nutraceutically acceptable excipient, wherein the weight ratio of Citrus bergamia extract (standardized in naringin 15%) to Pine tree extract (standardized in stanols 10%) is selected from:
10:1 to 1:10;
9:1 to 1:9;
8:1 to 1:8;
7:1 to 1:7;
6:1 to 1:6;
5:1 to 1:5;
4:1 to 1:4;
3:1 to 1:3; and
2:1 to 1:2.

preferably wherein the weight ratio of the Citrus bergamia extract (standardized in naringin 15%) to the Pine tree extract (standardized in stanols 10%) is 2:1 to 1:2.

The invention provides a pharmaceutical or nutraceutical composition comprising a combination of:
Citrus bergamia extract (standardized in naringin 15%) and
Pine tree extract (standardized in stanols 10%)

and a pharmaceutically or nutraceutically acceptable excipient, wherein the weight ratio of Citrus bergamia extract (standardized in naringin 15%) to Pine tree extract (standardized in stanols 10%) is selected from:
1:1 to 1:10;
1:1 to 1:9;
1:1 to 1:8;
1:1 to 1:7;
1:1 to 1:6;
1:1 to 1:5;
1:1 to 1:4;
1:1 to 1:3; and
1:1 to 1:2.

The invention provides a pharmaceutical or nutraceutical composition comprising a combination of:
naringin and
stanols

and a pharmaceutically or nutraceutically acceptable excipient.

The composition may additionally comprise one or more further active ingredients, selected from: HMG-CoA reductase inhibitors as fermented red rice (also known as red yeast rice), other botanical extracts for metabolic syndrome and cholesterolemia management as Berberis aristata (Berberine), Cynara scolymus extract, Cynara cardunculus extract, Allium sativum, Salvia miltiorrhiza, policosanol, green tea (Camellia sinensis) extract, annur apple (Melannurca campana) extract, Curcuma longa and curcuminoids, spirulina, chitosan, betaglucan, glucomannan, other active substances as coenzyme Q10, astaxanthin, folic acid, orthosiphon.

The invention provides a pharmaceutical composition comprising a combination of:
Citrus bergamia extract (standardized in naringin 15%) and
Pine tree extract (standardized in stanols 10%)

and a pharmaceutically or nutraceutically acceptable excipient.

The composition may additionally comprise one or more further active ingredients, selected from: HMG-CoA reductase inhibitors as fermented red rice (also known as red yeast rice), other botanical extracts for metabolic syndrome and cholesterolemia management as Berberis aristata (Berberine), Cynara scolymus extract, Cynara cardunculus extract, Allium sativum, Salvia miltiorrhiza, policosanol, green tea (Camellia sinensis) extract, annur apple (Melannurca campana) extract, Curcuma longa and curcuminoids, spirulina, chitosan, betaglucan, glucomannan, other active substances as coenzyme Q10, astaxanthin, folic acid, orthosiphon.

### Dosage

The combinations of the invention are useful in the treatment or prevention of hypercholesterolemia, hyperlipidemia, cardiovascular disease or metabolic syndrome.

The combination is generally administered to a subject in need of such administration, for example a human or animal, typically a human.

The combination will typically be administered in amounts that are therapeutically or prophylactically useful.

The compounds may be administered over a prolonged term to maintain beneficial therapeutic effects or may be administered for a short period only.

A typical daily dose of each components of the combination can be in the range from 100 pg to 100 mg per kg of body weight, more typically 5 ng to 25 mg per kg of bodyweight, and more usually 10 ng to 15 mg per kg (e.g. 10 ng to 10 to 20 mg, and more typically 1 μg per kg to 20 mg per kg, for example 1 μg to 10 mg per kg) per kg of bodyweight although higher or lower doses may be administered where required.

The components, naringin and stanols, of the combination may be administered orally in a range of doses, for example 0.1 to 750 mg, 1 to 225 mg, 2 to 120 mg, 5 to 75 mg, 2 to 30 mg, or 10 to 150 mg. Particular examples of daily doses are 10, 20, 50, 80, 150 and 300 mg.

In one embodiment, the combination or pharmaceutical or nutraceutical composition comprises from 15 mg to 65 mg naringin.

In one embodiment, the combination or pharmaceutical or nutraceutical composition comprises from 50 mg to 250 mg stanols.

In one embodiment, the combination or pharmaceutical or nutraceutical composition comprises:
from 15 mg to 65 mg naringin; and
from 50 mg to 250 mg stanols.

In one embodiment, the combination or pharmaceutical or nutraceutical composition comprises:
from 15 mg to 65 mg naringin; and
from 50 mg to 250 mg stanols, wherein the stanols comprise beta sitostanol and campestanol.

The components, Citrus bergamia (standardized in naringin 15%) and Pine tree extract (standardized in stanols 10%) of the combination may be administered orally in a range of doses, for example 0.1 to 5000 mg, 1 to 1500 mg, 2 to 800 mg, 5 to 500 mg, 2 to 20 mg, or 10 to 1000 mg.

Particular examples of daily doses are 100, 200, 500, 800, 1000 and 2500 mg.

In one embodiment, the combination or pharmaceutical or nutraceutical composition comprises 175 mg to 375 mg Citrus bergamia extract (standardized in naringin 15%).

In one embodiment, the combination or pharmaceutical or nutraceutical composition comprises 600 mg to 2500 mg Pine tree extract (standardized in stanols 10%).

In one embodiment, the combination or pharmaceutical or nutraceutical composition comprises:
175 mg to 375 mg Citrus bergamia extract (standardized in naringin 15%); and
600 mg to 2500 mg Pine tree extract (standardized in stanols 10%).

In one embodiment, the combination or pharmaceutical or nutraceutical composition comprises:
175 mg to 375 mg Citrus bergamia extract (standardized in naringin 15%); and
600 mg to 2500 mg Pine tree extract (standardized in stanols 10%), wherein the stanols comprise beta sitostanol and campestanol.

### Formulations

In one embodiment, one or more of the components of the combination are provided as oral dosage forms. Oral dosage forms include tablets (coated or uncoated), capsules (hard or soft shell), caplets, pills, lozenges, syrups, solutions, powders, granules, elixirs and suspensions, sublingual tablets, wafers or patches such as buccal patches.

In one embodiment, the compositions of the invention are provided as tablets.

Therefore, in one embodiment of the invention, at least one of the components (preferably all of the components) is presented in a tablet. In one embodiment, all components are presented in tablets, and in particular all components of the combination are presented in the same tablet i.e. the combination is administered in a unitary dose or fixed dose.

Typically, the tablet includes one or more pharmaceutically acceptable excipient. The pharmaceutically acceptable excipient can be selected from, for example, carriers (e.g. a solid, liquid or semi-solid carrier), adjuvants, diluents, fillers or bulking agents, granulating agents, coating agents, release-controlling agents, binding agents, disintegrants, lubricating agents, preservatives, antioxidants, buffering agents, suspending agents, thickening agents, flavouring agents, sweeteners, taste masking agents, stabilisers or any other excipients conventionally used in pharmaceutical compositions.

Preferably, the compositions of the invention are formulated with one or more pharmaceutically acceptable fillers or bulking agents.

Examples of excipients include dibasic calcium phosphate anhydrous, magnesium stearate, silicon dioxide, carboxymethylcellulose, crospovidone, and hydroxypropyl cellulose and maltodextrin.

In one embodiment, the compositions of the invention are provided in capsules.

Therefore, in one embodiment of the invention, at least one of the components (preferably all of the components) is presented in a capsule. In one embodiment, all of the components are presented in capsules, and in particular all components of the combination are presented in the same capsule i.e. the combination is administered in a unitary dose or fixed dose.

Typically, the capsule includes one or more pharmaceutically or nutraceutically acceptable excipient. The pharmaceutically or nutraceutically acceptable excipient can be selected from, for example, carriers (e.g. a solid, liquid or semi-solid carrier), adjuvants, diluents, fillers or bulking agents, granulating agents, coating agents, release-controlling agents, binding agents, disintegrants, lubricating agents, preservatives, antioxidants, buffering agents, suspending agents, thickening agents, flavouring agents, sweeteners, taste masking agents, stabilisers or any other excipients conventionally used in pharmaceutical compositions.

Examples of excipients include dibasic calcium phosphate anhydrous, magnesium stearate, silicon dioxide, maltodextrin, carboxymethylcellulose, crospovidone, and hydroxypropyl cellulose.

In one embodiment, the compositions of the invention are provided as granulates.

Therefore, in one embodiment of the invention, at least one of the components (preferably all of the components) is presented as a granulate. In one embodiment, all of the components are presented in a granulate, and in particular all components of the combination are presented in a single granulate i.e. the combination is administered in a unitary dose or fixed dose. The granulate may be packaged into a sachet or a stick pack.

The granulate may be prepared by dry or wet granulation techniques that are known in the art.

### FORMULATION EXAMPLES

| Tablet | mg/tab |
|---|---|
| Citrus bergamia extract | 187.5 |
| (of which naringin | 28) |
| Pine tree extract | 656.6 |
| (of which stanols | 66) |
| Microcrystalline cellulose | 331.9 |
| Silicon dioxide | 12 |
| Magnesium stearate | 12 |
| | 1200 |

| Tablet | mg/tab |
|---|---|
| Citrus bergamia extract | 187.5 |
| (of which naringin | 28) |
| Pine tree extract | 656.6 |
| (of which stanols | 66) |
| Microcrystalline cellulose | 220.9 |
| Hydroxypropyl cellulose | 48 |
| Crospovidone | 36 |
| Silicon dioxide | 12 |
| Magnesium stearate | 12 |
| Iron oxide | 27 |
| | 1200 |

| Granulate | mg/sachet |
|---|---|
| Citrus Bergamia extract | 375 |
| (of which naringin | 56) |
| Pine tree extract | 2222.0 |
| (of which stanols | 230) |
| D-Mannitol | 1334.4 |
| Flavour | 31 |
| Sucralose | 17.6 |
| Silicon dioxide | 20 |
| | 4000 |

| Granulate | mg/sachet |
|---|---|
| Citrus Bergamia extract | 375 |
| (of which naringin | 56) |
| Pine tree extract | 2222.0 |
| (of which stanols | 230) |
| D-Mannitol | 402.4 |
| Xylitol | 630 |
| Maize starch | 102 |
| Flavour | 31 |
| Sucralose | 17.6 |
| Citric acid | 180 |
| Stevia | 20 |
| Silicon dioxide | 20 |
| | 4000 |

### Examples

### Example 1 Synthesis

Citrus bergamia Extract

Bergamot (Citrus bergamia Risso & Poiteau) is a citrus fruit grown substantially only in restricted areas of Calabria and Sicily. The harvest period is from October to December. The Bergamot fruits are manually collected.

The Citrus bergamia extract is prepared by chromatographic adsorption followed by desorption using a solvent (e.g. water:ethanol 1:1). The bergamot juice is first microfiltered and then extracted by adsorption chromatography. The resins of the columns are washed with a solution of ethanol and water.

The resulting liquid is then concentrated at 40° C. under vacuum, and then combined with maltodextrin and silica. The resulting liquid is then subjected to a spray drying step and milled. The final homogenization takes place through a double conic blender and filling into a drum.

The chromatographic adsorption/microfiltration used to obtain the Citrus bergamia extract provides an extract with a high flavonoid content (40% w/w) which is particularly advantageous. The physical adsorption technique and the use of columns with a high number of theoretical plates make it possible to achieve a concentration of flavonoids that cannot be accessed with other known extraction techniques.

Citrus bergamia Extract Standardisation:

Based to the extraction process used, the Citrus bergamia extract includes the following active components:

| Component | Concentration of component (w/w %) |
|---|---|
| Naringin | 15 ± 5 |
| Neoeriocitrin | 10 ± 5 |
| Neohesperidin | 15 ± 5 |

### Pine Tree Extract

Pine tree extract is prepared by the following methodology:

### Plant Origin

From the tall oil pitch of Pinus elliottii and Pinus taeda. 100% from non-genetically modified pine.

### Extraction Technology

1. Saponification, from the tall oil pitch with caustic soda
2. Evaporation and neutral destillation
3. Crystallisation and filtration using solvents: hexane, ethanol and water
4. Drying and pastillation

### Pine Tree Extract Standardisation

Based on the extraction process used, the Pine tree includes the following active components:

| Component | Concentration of component (w/w %) |
|---|---|
| Beta sitostanol | 10 ± 5 |
| Campestanol | 2 ± 1 |

### Example 2: Cholesterol Reduction

### Materials and Methods

### Cells

Experiments were performed on Human Aortic Endothelial Cells (HAEC, Gibco). Cells were grown in Endothelial Cell Grown Medium (EGM 2-MV medium, Lonza) and were maintained at 37° C. in a humidified atmosphere of 5% CO₂. Ox-LDL Human LDL Copper Oxidized was purchased by (Cell Biolabs or by Cloud-Clone-Corp) and was used for cholesterol, quantification and lipid peroxidation evaluation.

### Measurement of Cell Viability

For cell viability HAEC were plated in 96 well/plate in their complete medium. After 12 hrs, HAEC were treated with the compounds for 24 hrs. Cell viability was evaluated with a colorimetric metabolic assay (MTS) (CellTiterAqueous, Promega). Absorption (490 nm) was monitored with Filtermax F5 Multi-Mode Microplate Reader (Molecular Devices, USA).

### Cholesterol Determination.

To evaluate cholesterol production by HAEC the Cholesterol/Cholesteryl Ester Assay Kit (Abcam) was used, which provides a simple colorimetric method for sensitive quantification of free cholesterol. Briefly, the HAEC monolayer was trypsinized and washed with PBS salt solution. Cells (1×10⁶) were washed with cold PBS, and lipid extraction was realized using 200 ml of Chloroform:Methanol (4:1) solution. The extract was centrifuged for 10 min at 15,000 g. Finally, samples were treated with cholesterol reaction mix and incubated for 60′ at 37° C. Optical density (595 nm) was observed with a F5 FilterMax microplate reader (Molecular Devices, US).

### Measurement of Lipid Peroxidation.

Lipid peroxidation was detected with Click-iT® Lipid Peroxidation Imaging Kit—Alexa Fluor® 488 leverages copper-catalyzed click chemistry and the linoleamide alkyne (LAA) reagent (alkyne-modified linoleic acid) for detection of lipid peroxidation-derived protein modifications in fixed cells. Click-iT® LAA (linoleamide alkyne) and pine oil and Citrus bergamia extract were added to cells and incubated for 60 min. Cells were washed three times with PBS.3. Cells fixed and permeabilized, and then blocked with 1% BSA. Click-iT® reaction cocktail was prepared, to which was added 125 μL/well, and incubated for 30 minutes at room temperature, protected from light. Cells were washed twice with 1% BSA, and twice more with PBS. The microplates were imaged and analyzed with a F5 FilterMax microplate reader.

### Results

### Cholesterol Determination

Data are expressed as Fold increase vs OxLDL

Data are the average of at least 3 repetitions of the experiment

### Concentrations Tested in Table 1:

CTRL: Control
Ox-LDL
S: Pine tree extract (standardized in stanols 10%): 1 μg/ml, 2 μg/ml
B: Citrus bergamia extract (standardized in naringin 15%): 1 μg/ml, 2 μg/ml

TABLE 1
Cholesterol reduction of Pine tree extract and Citrus bergamia
extract versus control

| | | | Ox-LDL + | Ox-LDL + | Ox-LDL + | Ox-LDL + |
|---|---|---|---|---|---|---|
| | CTRL | Ox-LDL | S 2 μg/ml | S 1 μg/ml | B 2 μg/ml | B 1 μg/ml |
| Mean | 0.617 | 1.000 | 0.638 | 0.618 | 0.873 | 0.956 |
| St. Dev. | 0.090 | 0.142 | 0.194 | 0.114 | 0.142 | 0.222 |

### Combinations Tested in Table 2:

S: Pine tree extract (standardized in stanols 10%):
B: Citrus bergamia extract (standardized in naringin 15%)
   S:B: 2:1 (2 μg/ml: 1 μg/ml)
   S:B: 1:2 (1 μg/ml: 2 μg/ml)
   S:B: 1:1 (2 μg/ml)

TABLE 2
Cholesterol reduction of Pine tree extract/
Citrus bergamia extract combination

| | Ox-LDL + | Ox-LDL + | Ox-LDL + |
|---|---|---|---|
| | S2:B1 μg/ml | S1:B2 μg/ml | S2:B2 μg/m |
| Mean | 0.384 | 0.435 | 0.389 |
| St. Dev. | 0.076 | 0.093 | 0.105 |
| P Value vs B | 0.0043 | 0.0061 | 0.0159 |
| P Value vs S | 0.0476 | 0.0333 | 0.2500 |

### Lipid Peroxidation

Data are expressed as Fold increase vs OxLDL

Data are the average of at least 3 repetitions of the experiment

### Concentrations Tested in Table 3:

CTRL: Control
Ox-LDL
S: Pine tree extract (standardized in stanols 10%): 1 μg/ml, 2 μg/ml
B: Citrus bergamia extract (standardized in naringin 15%): 1 μg/ml, 2 μg/ml

TABLE 3
Lipid peroxidation of Pine tree extract and Citrus bergamia
extract versus control

| | | | Ox-LDL + | Ox-LDL + | Ox-LDL + | Ox-LDL + |
|---|---|---|---|---|---|---|
| | CTRL | Ox-LDL | S 2 μg/ml | S 1 μg/ml | B 2 μg/ml | B 1 μg/ml |
| Mean | 1.000 | 4.323 | 1.563 | 1.641 | 2.030 | 2.045 |
| St. Dev. | 0.363 | 1.453 | 0.376 | 0.573 | 0.561 | 0.709 |

### Combinations Tested in Table 4:

S: Pine tree extract (standardized in stanols 10%):
B: Citrus bergamia extract (standardized in naringin 15%)
   S:B: 2:1 (2 μg/ml: 1 μg/ml)
   S:B: 1:2 (1 μg/ml: 2 μg/ml)

TABLE 4
Lipid peroxidation of Pine tree extract/
Citrus bergamia extract combination

| | Ox-LDL + | Ox-LDL + | Ox-LDL + |
|---|---|---|---|
| | S2:B1 μg/ml | S1:B2 μg/ml | S2:B2 μg/ml |
| Mean | 0.815 | 0.919 | 0.892 |
| St. Dev. | 0.222 | 0.315 | 0.449 |
| P Value vs B | 0.0047 | 0.0080 | 0.0140 |
| P Value vs S | 0.0490 | 0.0719 | 0.0490 |

### CONCLUSIONS

The results obtained show how the combinations provide:
A high reduction in the production of cholesterol compared to the values of the single substances.
A reduction in the production of cholesterol always higher than the control.
A high reduction in lipid peroxidation compared to the values of the single substances
A reduction in lipid peroxidation always higher than the control

1-17. (canceled)

18. A combination comprising naringin and stanols for use in the treatment or prevention of hypercholesterolemia, hyperlipidemia, cardiovascular disease, or metabolic syndrome.

19. The combination according to claim 18, wherein the wt ratio of naringin to stanols is from 10:1 to 1:10.

20. The combination according to claim 18, wherein the wt ratio of naringin to stanols is from 0.1:750 mg/dose to 750:0.1 mg/dose.

22. The combination according to claim 18, wherein naringin and stanols are administered simultaneously in a unitary formulation.

23. The combination according to claim 18, wherein naringin and stanols are administered in separate formulations either sequentially or simultaneously.

24. The combination according to claim 18, wherein the stanols comprise beta sitostanol, campestanol, or combinations therein.

25. A combination comprising Citrus bergamia extract (standardized in naringin 15%) and Pine tree extract (standardized in stanols 10%) for use in the treatment or prevention of hypercholesterolemia, hyperlipidemia, cardiovascular disease, or metabolic syndrome.

26. The combination according to claim 25, wherein the wt ratio of Citrus bergamia extract (standardized in naringin 15%) to Pine tree extract (standardized in stanols 10%) is from 10:1 to 1:10.

27. The combination according to claim 25, wherein the wt ratio of Citrus bergamia extract (standardized in naringin 15%) to Pine tree extract (standardized in stanols 10%) is from 0.1:5000 mg/dose to 5000:0.1 mg/dose.

28. The combination according to claim 25, wherein the Citrus bergamia extract (standardized in naringin 15%) to Pine tree extract (standardized in stanols 10%) are administered simultaneously in a unitary formulation.

29. The combination according to claim 25, wherein the Citrus bergamia extract (standardized in naringin 15%) to Pine tree extract (standardized in stanols 10%) are administered in separate formulations either sequentially or simultaneously.

30. The combination according to claim 25, wherein the Pine tree extract (standardized in stanols 10%) comprises beta sitostanol, campestanol, or combinations therein.

31. The combination according to claim 18, wherein the combination further comprises the HMG-CoA reductase inhibitor fermented red rice.

32. The combination according to claim 18, wherein the combination further comprises at least one substance selected from botanical extracts Berberis aristata (Berberine), Cynara scolymus extract, Cynara cardunculus extract, Allium sativum, Salvia miltiorrhiza, policosanol, green tea (Camellia sinensis) extract, annur apple (Melannurca campana) extract, Curcuma longa and curcuminoids, spirulina, chitosan, betaglucan, and glucomannan.

33. The combination according to claim 18, wherein the combination further comprises coenzyme Q10, astaxantine, folic acid, or orthosiphon.

34. The combination according to claim 18, wherein the combination does not comprise tocotrienols, hydroxytyrosol, or an extract comprising hydroxytyrosol.

35. The combination according to claim 18, wherein the combination is in the form of a solid oral composition comprising granulate, granules, grains, beads, or pellets.

36. The combination according to claim 18, wherein the combination is formulated as a capsule, tablet, or sachet.
